(19) United States
(12) Patent Application Publication
(10) Pub. No.: US 2023/0191138 A1
(43) Pub. Date: Jun. 22, 2023

(54) IMPLANTABLE MEDICAL DEVICE AND METHOD OF FORMING SAME

(71) Applicant: Medtronic, Inc., Minneapolis, MN (US)

(72) Inventors: Andrew J. Ries, Lino Lakes, MN (US); Pradipta K. Das, Plymouth, MN (US); Kelly A. DeVries, Lino Lakes, MN (US)

(21) Appl. No.: 17/877,303

(22) Filed: Jul. 29, 2022

Related U.S. Application Data
(60) Provisional application No. 63/291,681, filed on Dec. 20, 2021.

(51) Int. Cl. A61N 1/375; A61N 1/39

(52) U.S. Cl. CPC A61N 1/3754; A61N 1/3956

(57) ABSTRACT

Various embodiments of an implantable medical device and a system that includes such device are disclosed. The device includes a housing including a polymeric material, and an electronics module disposed within the housing and having a substrate, a power source disposed on the substrate, and circuitry disposed on the substrate and electrically connected to the power source. The device also includes a conformal coating disposed over at least a portion of the electronics module.

### CROSS-REFERENCE TO RELATED APPLICATION

The present application claims the benefit of U.S. Provisional Application No. 63/291,681, filed Dec. 20, 2021, which is incorporated herein by reference in its entirety.

### TECHNICAL FIELD

This disclosure generally relates to an implantable medical device.

### BACKGROUND

Implantable medical device systems such as implantable pacemakers can deliver pacing pulses to a patient's heart and monitor conditions of the patient's heart. In some examples, the implantable pacemaker includes an electronic assembly disposed within a housing and one or more electrical leads. The electronic assembly may, for example, be implanted in a small pocket in the patient's chest. The electrical leads can be coupled to the electronic assembly, which may include one or more electronic components that generate pacing pulses and/or sense cardiac electrical activity. The electrical leads may extend from the electronic assembly to a target site (e.g., an atrium and/or a ventricle) such that electrodes disposed on or within the electrical leads are positioned at the target site. The electronic assembly may provide electrical stimulation to the target site and/or monitor cardiac electrical activity at the target site via the electrodes.

A pacemaker typically has a hermetically sealed metal housing that encloses circuitry and a power source in a moisture free environment. A polymeric connector module can be connected to the metal housing, where the connector module provides a lead bore for insertion of a lead and one or more electrical contacts that are configured to electrically connect the inserted lead to circuitry disposed within the housing. The connector module can also include a setscrew block that is adapted to retain the lead within the lead bore.

### SUMMARY

The techniques of this disclosure generally relate to an implantable medical device and system that includes such device. The implantable medical device can include a housing and an electronics module disposed within the housing. A conformal coating can be disposed over at least a portion of the electronics module using any suitable technique or techniques such as atomic layer deposition. The conformal coating can, in one or more embodiments, seal the electronics module and prevent or slow ingress of contaminants such as bodily fluids that can damage components of the electronics module such as power sources and circuitry.

This disclosure includes without limitation the following clauses:

Clause 1: An implantable medical device that includes a housing including a polymeric material, and an electronics module disposed within the housing and including a substrate, a power source disposed on the substrate, and circuitry disposed on the substrate and electrically connected to the power source. The device further includes a conformal coating disposed over at least a portion of the electronics module.

Clause 2: The device of Clause 1, where the conformal coating covers substantially all of the electronics module.

Clause 3: The device of any one of Clauses 1-2, where the conformal coating completely encases the electronics module.

Clause 4: The device of any one of Clauses 1-3, where the power source is disposed on a first major surface of the substrate and the circuitry is disposed on a second major surface of the substrate.

Clause 5: The device of any one of Clauses 1-4, where the housing includes a first portion and second portion connected to the first portion.

Clause 6: The device of any one of Clauses 1-5, further including a desiccant disposed within the housing.

Clause 7: The device of any one of Clauses 1-6, further including a contact disposed within the housing and adapted to electrically connect the electronics module to a lead disposed within a lead bore of the housing that extends between a first end at an outer surface of the housing and a second end disposed within the housing.

Clause 8: The device of Clause 7, where the housing defines a first cavity and a second cavity, where the electronics module is disposed in the first cavity, where the contact is disposed in the second cavity, and further where the second end of the lead bore is connected to the second cavity.

Clause 9: The device of any one of Clauses 1-8, further including a setscrew block disposed within the housing.

Clause 10: The device of any one of Clauses 1-9, where the conformal coating includes an atomic or molecular layer deposited conformal coating.

Clause 11: The device of any one of Clauses 1-9, where the conformal coating includes a vapor deposited conformal coating.

Clause 12: The device of any one of Clauses 1-9, where the conformal coating includes a dip coated conformal coating.

Clause 13: The device of any one of Clauses 1-12, where the circuitry further includes an antenna.

Clause 14: The device of any one of Clauses 1-13, further including a polymeric filler material disposed within the housing.

Clause 15: The device of any one of Clauses 1-14, where the conformal coating includes a polymeric material.

Clause 16: The device of Clause 15, where the conformal coating includes a parylene material.

Clause 17: The device of any one of Clauses 1-14, where the conformal coating includes an oxide material.

Clause 18: The device of Clause 17, where the oxide material includes at least one of titanium oxide or aluminum oxide.

Clause 19: The device of any one of Clauses 1-14, where the conformal coating includes two or more layers.

Clause 20: The device of Clause 19, where the conformal coating includes alternating layers of an oxide material and a parylene material.

Clause 21: The device of Clause 19, where the conformal coating includes alternating layers of a polymer material and an oxide material.

Clause 22: The device of Clause 1, where the conformal coating includes a first portion disposed over the power source and a second portion disposed over the circuitry.

Clause 23: The device of Clause 22, where the first portion of the conformal coating includes a first material and the second portion of the conformal coating includes a second material different from the first material.

Clause 24: An implantable medical device system that includes an implantable medical device and a lead. The implantable medical device includes a housing including a polymeric material, and an electronics module disposed within the housing and including a substrate, a power source disposed on the substrate, and circuitry disposed on the substrate and electrically connected to the power source. The device further includes a conformal coating disposed over at least a portion of the electronics module. At least a portion of the lead is adapted to be disposed within a lead bore of the housing and electrically connected to the electronics module.

Clause 25: The system of Clause 24, where the lead bore of the implantable medical device includes a contact that is electrically connected to the electronics module by a conductor, where a lead contact of the lead is adapted to be electrically connected to the contact of the lead bore when the at least a portion of the lead is disposed within the lead bore.

Clause 26: The system of Clause 25, where the housing defines a first cavity and a second cavity, where the electronics module disposed in the first cavity, where the contact is disposed in the second cavity, and further where the lead bore extends from an outer surface of the housing and into the second cavity.

Clause 27: The system of any one of Clauses 24-26, where the implantable medical device is an implantable defibrillator.

Clause 28: The system of any one of Clauses 24-27, where the implantable medical device is a neuromodulation device.

Clause 29: The system of any one of Clauses 24-28, where the conformal coating covers substantially all of the electronics module.

Clause 30: The system of any one of Clauses 24-29, where the conformal coating completely encases the electronics module.

Clause 31: The system of any one of Clauses 24-30, where the power source is disposed on a first major surface of the substrate and the circuitry is disposed on a second major surface of the substrate.

Clause 32: The system of any one of Clauses 24-31, where the housing includes a first portion and second portion connected to the first portion.

Clause 33: The system of any one of Clauses 24-32, further including a desiccant disposed within the housing.

Clause 34: The system of any one of Clauses 24-33, further including a setscrew block disposed within the housing.

Clause 35: The system of any one of Clauses 24-34, where the conformal coating includes an atomic or molecular layer deposited conformal coating.

Clause 36: The system of any one of Clauses 24-35, where the conformal coating includes a vapor deposited conformal coating.

Clause 37: The system of any one of Clauses 24-36, where the circuitry further includes an antenna.

Clause 38: The system of any one of Clauses 24-37, further including a polymeric filler material disposed within the housing.

Clause 39: The system of any one of Clauses 24-38, where the conformal coating includes a polymeric material.

Clause 40: The system of Clause 39, where the conformal coating includes a parylene material.

Clause 41: The system of any one of Clauses 24-38, where the conformal coating includes an oxide material.

Clause 42: The system of Clause 41, where the oxide material includes at least one of titanium oxide or aluminum oxide.

Clause 43: The system of any one of Clauses 24-38, where the conformal coating includes two or more layers.

Clause 44: The system of Clause 43, where the conformal coating includes alternating layers of an oxide material and a parylene material.

Clause 45: The system of Clause 43, where the conformal coating includes alternating layers of an oxide material.

Clause 46: The system of Clause 44, where the conformal coating includes alternating layers of a polymer material and an oxide material.

Clause 47: The system of any one of Clauses 24-46, where the conformal coating includes a first portion disposed over the power source and a second portion disposed over the circuitry.

Clause 48: The system of Clause 47, where the first portion of the conformal coating includes a first material and the second portion of the conformal coating includes a second material different from the first material.

Clause 49: A method of forming an implantable medical device. The method includes disposing a conformal coating over at least a portion of an electronics module, where the electronics module includes a power source and circuitry disposed on a substrate, where the circuitry is electronically connected to the power source; and disposing the electronics module in a polymeric housing.

Clause 50: The method of Clause 49, where disposing the conformal coating includes atomic layer depositing the conformal coating over the at least a portion of the electronics module.

Clause 51: The method of Clause 49, where disposing the conformal coating includes vapor depositing the conformal coating over the at least a portion of the electronics module.

Clause 52: The method of Clause 49, where disposing the conformal coating includes disposing a first portion of the conformal coating over the power source and disposing a second portion of the conformal coating over the circuitry.

Clause 53: The method of Clause 52, where the first portion includes parylene and second portion includes an oxide.

Clause 54: The method of any one of Clauses 49-53, where the conformal coating includes two or more layers of alternating material.

Clause 55: The method of any one of Clauses 49-54, where disposing the conformal coating includes disposing the conformal coating over substantially all of the electronics module.

Clause 56: The method of any one of clauses 49-55, further including disposing a polymeric filler material within the housing.

Clause 57: The method of Clause 56, where disposing the polymeric filler material includes disposing the polymeric filler material through an opening disposed in the housing.

Clause 58: The method of any one of clauses 49-57, further including disposing a lead bore in the housing that extends from an outer surface of the housing into the housing.

Clause 59: The method of Clause 58, further including disposing a contact within the lead bore that is electrically connected to the electronics module.

Clause 60: The method of any one of Clauses 58-59, further including disposing a portion of a lead into the lead bore such that a lead contact of the lead is electrically connected to the contact disposed within the lead bore.

Clause 61: The method of any one of Clauses 58-60, further including disposing a setscrew block within the lead bore.

Clause 62: The method of any one of Clauses 49-61, where the housing includes a first portion and a second portion, where disposing the electronics module within the housing includes disposing the electronics module in the first portion of the housing and connecting the second portion of the housing to the first portion.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic view of one embodiment of an implantable medical device system disposed within a patient.

FIG. 2 is a schematic perspective view of the implantable medical device system of FIG. 1.

FIG. 3 is a schematic cross-section view of an implantable medical device of the implantable medical device system of FIG. 1.

FIG. 4 is a rear view of the implantable medical device of FIG. 3.

FIG. 5 is a schematic view of a portion of a housing of the implantable medical device of FIG. 3 with circuitry of the device removed

FIG. 6 is a schematic cross-section view of an electronics module of the implantable medical device of FIG. 3 with a conformal coating disposed over at least a portion of the module.

FIG. 7 is a schematic plan view of a power source of the electronics module of FIG. 6.

FIG. 8 is a schematic plan view of circuitry of the electronics module of FIG. 6.

FIG. 9 is a schematic cross-section view of the implantable medical device system of FIG. 1.

FIG. 10 is a schematic cross-section view of a portion of one embodiment of a multilayer conformal coating.

FIG. 11 is a flowchart of one method of forming the implantable medical device system of FIG. 1.

### DETAILED DESCRIPTION

The techniques of this disclosure generally relate to an implantable medical device and system that includes such device. The implantable medical device can include a housing and an electronics module disposed within the housing. A conformal coating can be disposed over at least a portion of the electronics module using any suitable technique or techniques such as atomic layer deposition. The conformal coating can, in one or more embodiments, seal the electronics module and prevent or slow ingress of contaminants such as bodily fluids that can damage components of the electronics module such as power sources and circuitry.

Implantable medical devices such as pacemakers can be disposed within a body of patient and provide various types of treatments to the patient through delivery of electric or other types of signals. Because it is disposed within the body, these devices are exposed to fluids or tissue that may be directed into a housing of the device and potentially damage or contaminate electronics or power sources disposed within the housing.

To prevent ingress of these fluids, the housing can be hermetically sealed; however, certain types of materials such as titanium or other metals may be required to form such hermetically sealed housings. These materials can be expensive, and techniques that can be utilized to seal these materials together can also be expensive and present various technical challenges. Further, in circumstances where an implantable medical device may be for short term use, expensive materials such as titanium may not be desirable.

One or more embodiments of implantable medical devices described herein may include a housing that includes one or more polymeric materials that may be less expensive than materials that are typically used for such devices. Such polymeric housings may not need to provide a hermetically sealed enclosure as a conformal coating can be disposed over at least a portion of circuitry and power sources of the device that are disposed within the housing. Such conformal coating can be adapted to prevent ingress of fluids or other contaminants onto such circuitry and power sources that can damage them.

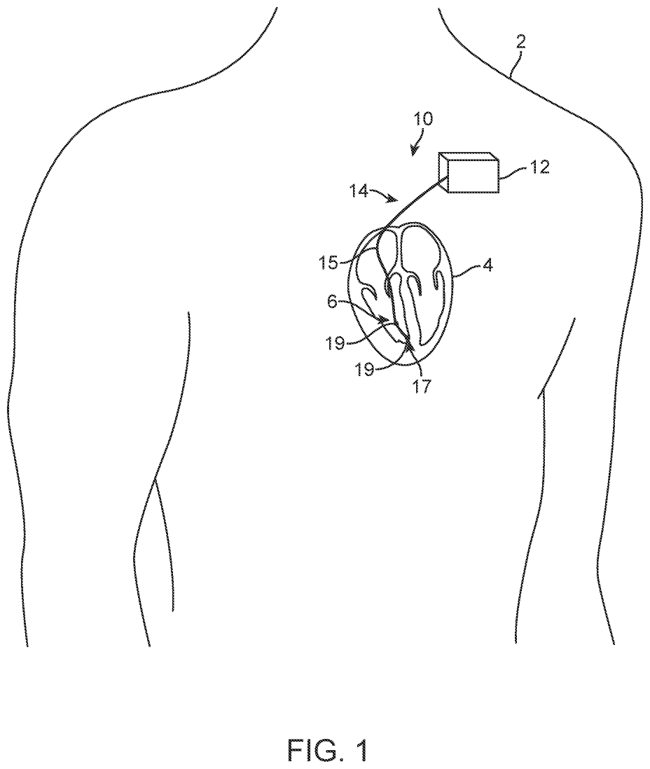

FIG. 1 is a schematic view of one embodiment of an implantable medical device system 10 disposed within a body of a patient 2. The system 10 includes an implantable medical device 12 and a lead 14 connected to the device. The system 10 can function as a single chamber, e.g., ventricular, pacemaker, as illustrated by the example of FIG. 1, or as a dual-chamber pacemaker that delivers pacing to a heart 4 of the patient 2.

As shown in the embodiment illustrated in FIG. 1, the lead 14 includes an elongated lead body 15 with a distal portion 17. The distal portion 17 is positioned at a target site 6 within a heart 4 of the patient 2. Distal portion 17 may include one or more electrodes 19. The target site 6 can be located at a wall of a ventricle of the heart 4. Further, the lead 14 can include any suitable lead, e.g., a bipolar or multipolar lead.

A clinician can maneuver distal portion 17 through the vasculature of the patient 2 to position the distal portion 17 at or near the target site 6. For example, the clinician may guide the distal portion 17 through the SVC to the target site 6 on or in a ventricular wall of the heart 4, e.g., at the apex of the right ventricle as illustrated in FIG. 1. In one or more embodiments, other pathways or techniques may be used to guide the distal portions 17 into other target implant sites within the body of the patient 2. The system 10 may include a delivery catheter and/or outer member (not shown), and the lead 14 can be guided and/or maneuvered within a lumen of the delivery catheter in order to approach the target site 6.

The lead 14 can include one or more electrodes 19 adapted to be positioned on, within, or near cardiac tissue at or near the target site 6. In one or more embodiments, the electrodes 19 are adapted to function as electrodes to, e.g., provide pacing to the heart 4. The electrodes 19 can be electrically connected to conductors (not shown) extending through the lead body 15. In one or more embodiments, the conductors are electrically connected to therapy delivery circuitry of the implantable medical device 12, with the therapy delivery circuitry adapted to provide electrical signals through the conductor to electrodes 19. The electrodes 19 can conduct the electrical signals to the target tissue of heart 4, causing the cardiac muscle, e.g., of the ventricles, to depolarize and, in turn, contract at a regular interval. The electrodes 19 can also be connected to sensing circuitry of IMD 12 via the conductors, and the sensing circuitry may sense activity of heart 4 via the electrodes 19. Such electrodes 19 may have various shapes such as tines, helices, screws, rings, and so on. Again, although a bipolar configuration of lead 14 including two electrodes 19 is illustrated in FIG. 1, in other embodiments IMD 12 can be coupled to leads including different numbers of electrodes, such as one electrode, three electrodes, or four electrodes.

In one or more embodiments, IMD 12 includes electronic circuitry contained within a housing where the circuitry may be adapted to deliver cardiac pacing. In the example of FIG. 1, the electronic circuitry within IMD 12 can include therapy delivery circuitry electrically coupled to electrodes 19. The electronic circuitry within IMD 12 can also include sensing circuitry configured to sense electrical activity of heart 4 via electrodes 19. The therapy delivery circuitry can be configured to administer cardiac pacing via the electrodes 19, e.g., by delivering pacing pulses in response to expiration of a timer and/or in response to detection of the activity (or absence thereof) of heart.

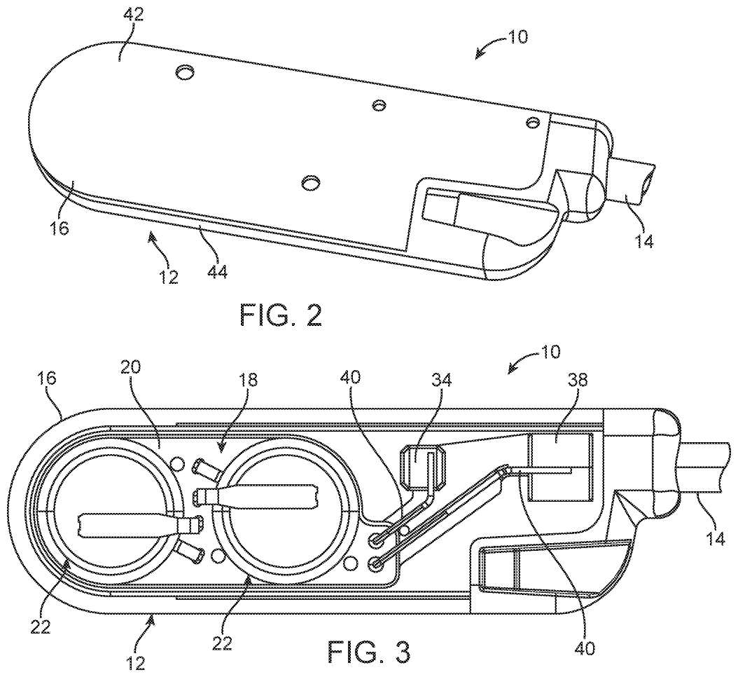

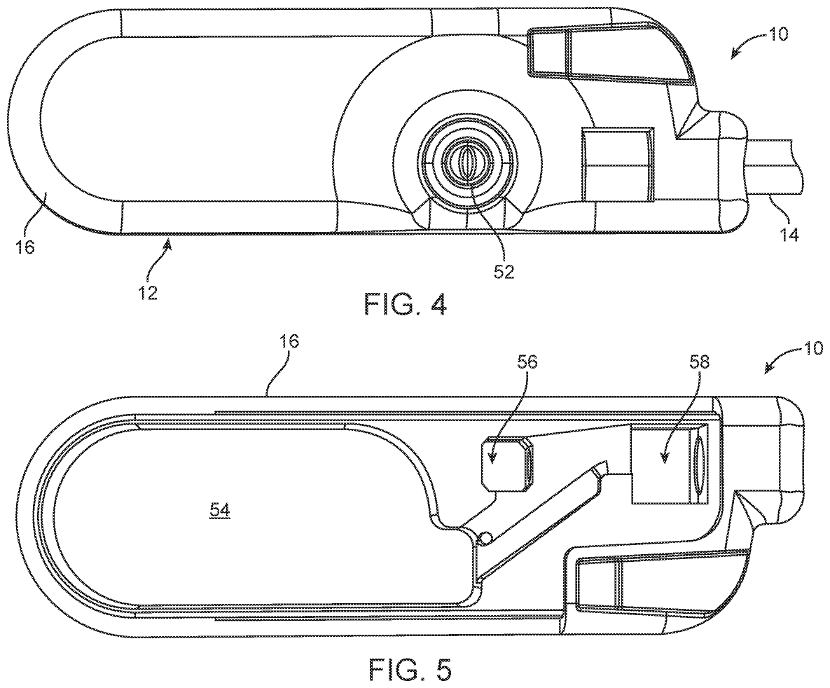

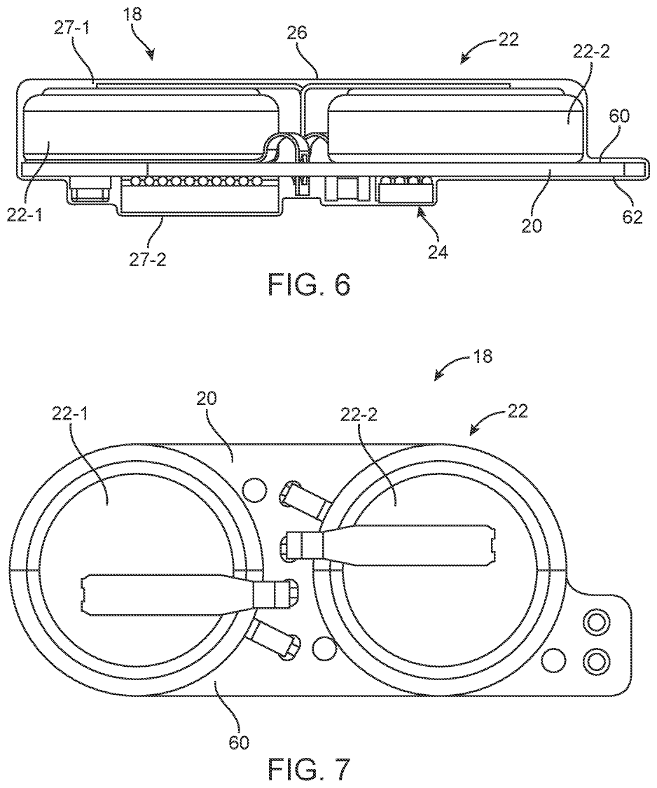

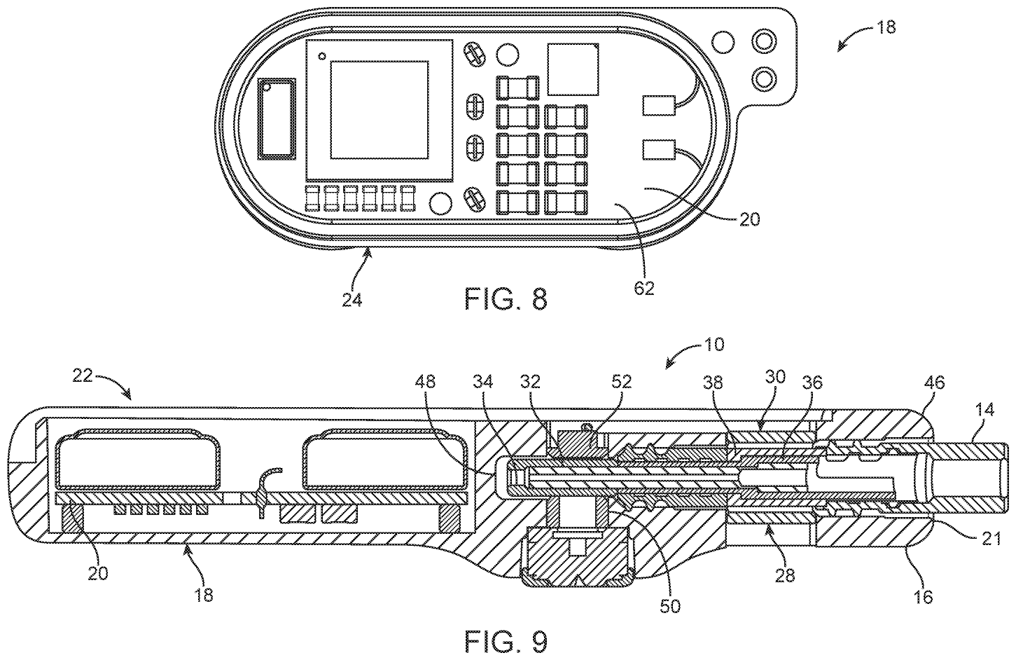

FIGS. 2-9 are various views of one embodiment of the implantable medical device system 10 of FIG. 1. The implantable medical device 12 includes a housing 16 and an electronics module 18 disposed within the housing. As shown in FIG. 6, the electronics module 18 can include a substrate 20, a power source 22 disposed on the substrate, and circuitry 24 also disposed on the substrate and electrically connected to the power source. The implantable medical device 12 also includes a conformal coating 26 (FIG. 6) disposed over at least a portion of the electronics module 18. As shown, e.g., in FIG. 9, at least a portion 28 of the lead 14 is adapted to be disposed within a lead bore 30 of the housing 16 and electrically connected to the electronics module 18.

The implantable medical device 12 of the system 10 can include any suitable medical device that is adapted to be implanted within a body of a patient. In one or more embodiments, the device 12 can be a pacemaker. Further, in one or more embodiments, the device 12 can be a leadless cardiac monitor. The device 12 can include any other suitable medical devices such as at least one of a defibrillator, LVAD, neuromodulation device, or drug pump.

The system 10 also includes the lead 14 that is adapted to be electrically connected to the electronics module 18. The lead 14 can include any suitable lead, e.g., pacing, defibrillation or nerve stimulation lead in an industry standard or custom format, etc. Although depicted as including one lead 14, the system 10 can include any suitable number of leads that are electrically connected to the electronics module 18. The lead 14 can be electrically connected to the electronics module 18 using any suitable technique or techniques. In one or more embodiments, at least a portion 28 of the lead 14 can be disposed within the lead bore 30 of the housing 16 such that a lead contact 32 is electrically connected to contact 34 disposed within the lead bore 30 when the portion 28 of the lead 14 is disposed within the lead bore. In one or more embodiments, the lead 14 can include a second lead contact 36 that is adapted to electrically connect the lead 14 to a second contact 38 disposed within the lead bore 30 when the portion 28 of the lead is disposed within the lead bore. The contact 34 and the second contact 38 can be electrically connected to the electronics module 18 by conductors 40 as is shown in FIG. 3.

The housing 16 of the device 12 can take any suitable shape or shapes and have any suitable dimensions. Further, the housing 16 can include any suitable material or materials, e.g., at least one of a metallic, polymeric, or inorganic material. Suitable materials for the housing 16 can include at least one of titanium (e.g., any suitable grade such as grade 5 titanium), stainless steel, polymer, ceramic, glass, or combinations thereof such as laminates, composites, or miscible blends or mixtures. In one or more embodiments, the housing 16 can include any suitable polymeric material or materials, e.g., at least one of epoxy, polyurethane, silicone, polyolefin, acrylic polymer, polyester, polyetheletherketone, polysulfone, polymethylene oxide, or polyvinyl, or combinations thereof.

The housing 16 can be a unitary housing. In one or more embodiments, the housing 16 can include two or more portions that are connected using any suitable technique or techniques, e.g., welding, mechanically fastening, adhering, thermal bonding, diffusion bonding, laser-assisted diffusion bonding, solvent bonding, over-molding, etc. For example, the housing 16 can include a first portion 42 and a second portion 44 (FIG. 2). Although illustrated in FIG. 2 as being transparent for sake of clarity, the first portion 42 can instead be opaque, translucent, etc. Similarly, the second portion 44 can be transparent, opaque, translucent, etc. The first portion 42 can be connected to the second portion 44 using any suitable technique or techniques. Further, the housing 16 can be formed using any suitable technique or techniques, e.g., molding, thermoforming, laminating, over-molding, casting, insert molding, etc. The first and second portions 42, 44 can include the same material. In one or more embodiments, the first portion 42 includes a material that is different from the material utilized to form the second portion 44. In one or more embodiments, the first and second portions 42, 44 can each include a polymeric material.

The housing 16 can include any suitable ports or receptacles that can connect the device to external components or systems. For example, the housing 16 can include the lead bore 30 disposed in any suitable portion or portions of the housing. As shown in FIG. 9, the lead bore 30 extends between a first end 46 and a second end 48. The lead bore 30 can extend from an outer surface 21 and the second end is disposed within the housing, e.g., within a second cavity 56 of the housing 16. The lead bore 30 can take any suitable shape or shapes and have any suitable dimensions. The housing 16 can also include a setscrew bore 50 disposed in any suitable portion or portions of the housing that is adapted to receive a setscrew block 52. The setscrew bore 50 can take any suitable shape or shapes and have any suitable dimensions. The setscrew block 52 can include any suitable fastener that is adapted to retain the lead 14 in the lead bore 30.

An external coating or coatings can be disposed over one or more portions of the housing 16 using any suitable technique. The external coating can include any suitable material or materials, e.g., a biocompatible material. Suitable biocompatible coatings can include at least one of a metallic, ceramic, or polymeric material. Suitable metallic materials include a sputter metallic coating or foil. Further, suitable ceramic materials include at least one of aluminum oxide, glass, mica, titanium oxide, titanium nitride, vanadium oxide, niobium oxide, zirconium oxide, hafnium oxide, silicon oxide, or silicon nitride. And suitable polymer materials include at least one of parylene, silicone, acrylic, polyurethan, or epoxy. In one or more embodiments, the coating can include a multilayer coating having any suitable material or materials, e.g., at least one of epoxy, parylene, polyimide, silicone, acrylic, or vinyl.

As shown in FIG. 5, which is a schematic plan view of the system 10 with the electronics module 18, lead 14, and first portion 42 of the housing 16 remove for clarity, the housing defines a first cavity 54 and the second cavity 56. The electronics module 18 can be disposed in the first cavity 54, and the contact 34 can be disposed in the second cavity 56. The housing 16 can further define a third cavity 58 within which the second contact 38 can be disposed.

The electronics module 18 can include the substrate 20, the power source 22 disposed on the substrate, and circuitry 24 disposed on the substrate and electrically connected to the power source. The substrate 20 can include any suitable material and have any suitable dimensions. As shown in FIG. 6, the substrate 20 includes a first major surface 60 and a second major surface 62.

Disposed on the substrate 20 is the power source 22, which can include any suitable power source or sources, e.g., one or more batteries. The power source 22 includes a first power source 22-1 and a second power source 22-2 (collectively power source 22). Although depicted as including two power sources, the power source 22 can include any suitable number of power sources, e.g., one, two, three, four, five, or more power sources. The power source 22 can be disposed on any suitable portion or portions of the substrate 20, e.g., on the first major surface 60, the second major surface 62, or on both the first and second major surfaces. In one or more embodiments, the power source 22 can be disposed in the housing 16 in any suitable location without being disposed on a substrate.

Also disposed on the substrate 20 is the circuitry 24. Such circuitry 24 can include any suitable device or component, e.g., at least one of a capacitor, transistor, integrated circuit including a controller or multiplexer, sensor, accelerometer, inductive charging coil, antenna, optical components such as emitters and detectors, etc. Such components can be electrically connected to the power source or one or more components using any suitable techniques or techniques. Further, the circuitry 24 can be disposed on any suitable portion of the substrate, e.g., on the first major surface 60, the second major surface 62, or on both the first and second major surfaces. In one or more embodiments, one or more components of the circuitry 24 can be disposed apart from one or more additional components of the circuitry within or on the housing 16 and electrically connected to the power source 22 and one or more additional components using any suitable technique or techniques.

Disposed over at least a portion of the electronics module 18 is the conformal coating 26. As used herein, the term “conformal” means that a layer or layers can be disposed on an element or component such that the layer or layers take the shape of the element or component. The coating 26 can be disposed over any suitable portion or portions of the electronics module 18. In one or more embodiments, the conformal coating 26 covers substantially all of the electronics module 18. Further, in one or more embodiments, the conformal coating 26 completely encases the electronics module 18, i.e., the electronics module is completely covered by the conformal coating such that electronics module is sealed by the coating.

The conformal coating 26 can include any suitable material or materials, e.g., at least one of inorganic, organic, or ceramic material. In one or more embodiments, the conformal coating 26 can include one or more polymeric materials, e.g., at least one of parylene, silicone, or urethane. In one or more embodiments, silicone or polyurethane can be thinned with a solvent to form a conformal coating composition, and dip coating can be utilized to apply the composition to form the conformal coating 26. In one or more embodiments, the conformal coating can include one or more oxide materials, e.g., at least one of aluminum oxide, tantalum oxide, or hafnium oxide.

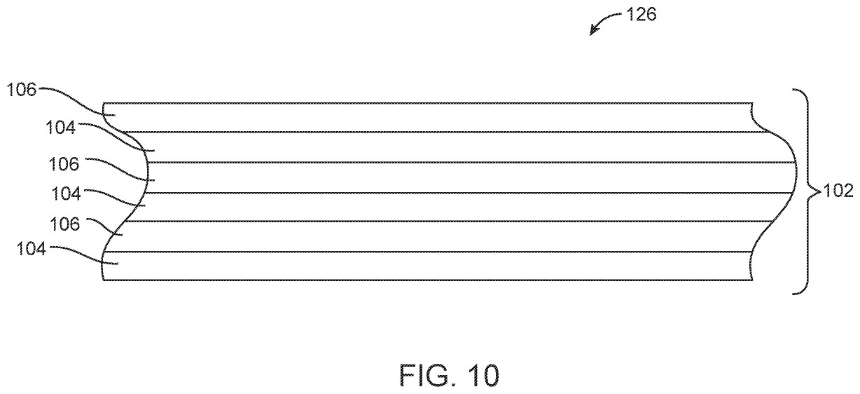

The conformal coating 26 can be a single layer or include two or more layers. For example, FIG. 10 is a schematic cross-section view of a portion of another embodiment of a conformal coating 126. All of the design considerations and possibilities regarding the conformal coating 26 of FIGS. 2-9 apply equally to the conformal coating 126 of FIG. 10. The coating 126 can be utilized with any suitable implantable medical device, e.g., implantable medical device 12 of FIGS. 1-9.

The coating 126 includes multiple layers 102 of material. In one or more embodiments, the coating 126 includes first layers 104 alternating with second layers 106. The first and second layers 104, 106 can be arranged in any suitable arrangement. Further, the coating 126 can include one or more third layers that are arranged in any suitable pattern with the first and second layers 104, 106. Any suitable number of layers can be utilized for the conformal coating 126. In one or more embodiments, each layer of the conformal coating 126 can include the same material. In one or more embodiments, the conformal coating 126 can include one or more layers that are different from one or more additional layers of the conformal coating. In one or more embodiments, the conformal coating 126 can include alternating layers of an oxide material and a polymer material, e.g., an oxide material and parylene. In one or more embodiments, the conformal coating 126 can include alternating layers of an oxide material. And in one or more embodiments, the conformal coating 126 can include alternating layers of an oxide material with a cap layer of parylene or other polymer coating.

Returning to FIG. 6, the conformal coating 26 can include two or more different materials disposed in different portions of the layer. For example, the conformal coating 26 can include a first portion 27-1 disposed over the power source 22 and a second portion 27-2 disposed over the circuitry 24 as shown in FIG. 6. The first portion 27-1 can include a first material and the second portion 27-2 can include a second material. In one or more embodiments, the first material is the same as the second material. In one or more embodiments, the first material of the first portion 27-1 can include a material that is different from the second material of the second portion 27-2.

Any suitable technique or techniques can be utilized to dispose the conformal coating 26 on one or more portions of the electronics module 18. For example, the conformal coating 26 can be an atomic layer deposited conformal coating. Such conformal coating can be disposed on at least a portion of the electronics module using any suitable atomic layer deposition techniques, e.g., one or more of the techniques described in U.S. Patent Publication No. 2007/0250142 to Francis et al. and entitled ATOMIC LAYER DEPOSITION COATINGS FOR IMPLANTABLE MEDICAL DEVICES. Further, the conformal coating 26 can be a vapor deposited conformal coating. Any suitable vapor deposition techniques can be utilized to dispose the conformal coating 26 on the electronics module 18.

The implantable medical device 12 can further include any suitable materials disposed within the housing 16. For example, a desiccant can be disposed in any suitable location within the housing 16, e.g., in the first cavity 54 of the housing. The desiccant can include any suitable material or materials that can absorb moisture present within the housing 16, e.g., at least one of a molecular sieve, silica gel, or a combination of silicone elastomer mixed with one of the desiccant materials.

Further, one or more polymeric filler materials can be disposed in any suitable location within the housing 16, e.g., in the first cavity 54. The polymeric filler material can include any suitable material or materials, e.g., at least one of a medical adhesive, a medical grade silicone, an epoxy, or a polyurethane.

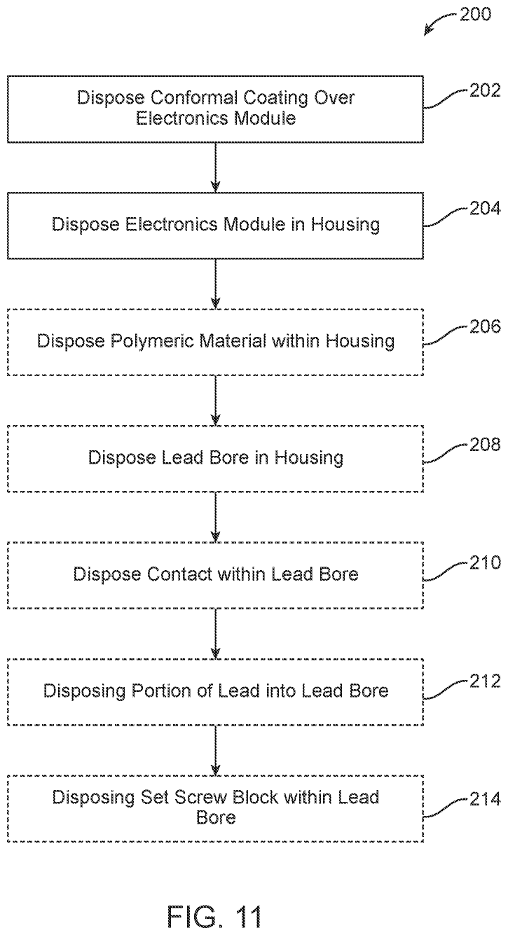

Any suitable technique or techniques can be utilized to form the implantable medical device 12. For example, FIG. 11 is a flowchart of one embodiment of a method 200 for forming an implantable medical device. Although described in regard to implantable medical device 12 of FIGS. 1-9, the method 200 can be utilized to form any suitable implantable medical device.

At 202, the conformal coating 26 is disposed over at least a portion of the electronics module 18 using any suitable technique or techniques, e.g., atomic layer deposition, vapor deposition, etc. In one or more embodiments, a first portion of the conformal coating can be disposed over the power source 22 and a second portion of the coating can be disposed over the circuitry 24. In one or more embodiments, the conformal coating can be disposed over substantially all of the electronics module 18. In one or more embodiments, one or more portions of the conductors 40 that connect the electronics module 18 to contacts 34, 38 are left uncovered by the conformal coating 26 such that they extend through the coating from the module to the contacts.

The electronics module 18 can be disposed within the polymeric housing 16 at 204. Further, at 206, polymeric filler material can optionally be disposed within the housing 16 through an opening disposed in the housing to fill space between the electronics module 18 and the cavity 54 and other spaces within the housing as is desired. Any suitable technique or techniques can be utilized to dispose such polymeric filler material into the housing.

At 208, the lead bore 30 can be disposed in the housing 16. In one or more embodiments, the housing 16 is injection molded to provide the lead bore 30. Further, at 210, one or both contacts 34, 38 can be disposed within the lead bore 30 and electrically connected to the electronics module 18 by conductors 40. At least a portion of the lead 14 can optionally be disposed within the lead bore 30 at 212 using any suitable technique or techniques. Further, the setscrew block 52 can optionally be disposed within a setscrew bore 50 at 214.

If the housing 16 includes two or more portions, such portions can be connected using any suitable technique or techniques at 216 to enclose the electronics module 18 and contacts 34, 38 within the housing. Any suitable technique or techniques can be utilized to connect the portions of the housing 16. In one or more embodiments, the housing 16 can include one or more holes that serve as gates and vents (not shown) to back fill the housing with any suitable material such as medical adhesive or epoxy to reduce or eliminate air voids within the housing 16 and to provide mechanical strength to the internal components. Such vents can be backfilled with any suitable material to seal the vents. In one or more embodiments, the back fill material can include an adhesive that can connect together two or more components disposed within the housing 16.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An implantable medical device comprising:
a housing comprising a polymeric material;
an electronics module disposed within the housing and comprising a substrate, a power source disposed on the substrate, and circuitry disposed on the substrate and electrically connected to the power source; and
a conformal coating disposed over at least a portion of the electronics module.

2. The device of claim 1, wherein the conformal coating covers substantially all of the electronics module.

3. The device of claim 1, wherein the power source is disposed on a first major surface of the substrate and the circuitry is disposed on a second major surface of the substrate.

4. The device of claim 1, further comprising a contact disposed within the housing and adapted to electrically connect the electronics module to a lead disposed within a lead bore of the housing that extends between a first end at an outer surface of the housing and a second end disposed within the housing.

5. The device of claim 4, wherein the housing defines a first cavity and a second cavity, wherein the electronics module is disposed in the first cavity, wherein the contact is disposed in the second cavity, and further wherein the second end of the lead bore is connected to the second cavity.

6. The device of claim 1, wherein the conformal coating comprises an atomic or molecular layer deposited conformal coating.

7. The device of claim 1, wherein the conformal coating comprises an oxide material comprising at least one of titanium oxide or aluminum oxide.

8. The device of claim 1, wherein the conformal coating comprises two or more layers.

9. The device of claim 8, wherein the conformal coating comprises alternating layers of an oxide material and a parylene material.

10. The device of claim 1, wherein the conformal coating comprises a first portion disposed over the power source and a second portion disposed over the circuitry, wherein the first portion of the conformal coating comprises a first material and the second portion of the conformal coating comprises a second material different from the first material.

11. An implantable medical device system comprising an implantable medical device and a lead, wherein the implantable medical device comprises:
a housing comprising a polymeric material;
an electronics module disposed within the housing and comprising a substrate, a power source disposed on the substrate, and circuitry disposed on the substrate and electrically connected to the power source; and
a conformal coating disposed over at least a portion of the electronics module;
wherein at least a portion of the lead is adapted to be disposed within a lead bore of the housing and electrically connected to the electronics module.

12. The system of claim 11, wherein the lead bore of the implantable medical device comprises a contact that is electrically connected to the electronics module by a conductor, wherein a lead contact of the lead is adapted to be electrically connected to the contact of the lead bore when the at least a portion of the lead is disposed within the lead bore.

13. The system of claim 12, wherein the housing defines a first cavity and a second cavity, wherein the electronics module disposed in the first cavity, wherein the contact is disposed in the second cavity, and further wherein the lead bore extends from an outer surface of the housing and into the second cavity.

14. The system of claim 11, wherein the implantable medical device is an implantable defibrillator.

15. The system of claim 11, wherein the conformal coating covers substantially all of the electronics module.

16. The system of claim 11, wherein the power source is disposed on a first major surface of the substrate and the circuitry is disposed on a second major surface of the substrate.

17. The system of claim 11, wherein the conformal coating comprises an atomic or molecular layer deposited conformal coating.

18. A method of forming an implantable medical device comprising:
disposing a conformal coating over at least a portion of an electronics module, wherein the electronics module comprises a power source and circuitry disposed on a substrate, wherein the circuitry is electronically connected to the power source; and
disposing the electronics module in a polymeric housing.

19. The method of claim 18, wherein disposing the conformal coating comprises disposing a first portion of the conformal coating over the power source and disposing a second portion of the conformal coating over the circuitry.

20. The method of claim 18, wherein disposing the conformal coating comprises disposing the conformal coating over substantially all of the electronics module.
